(19) United States
(12) Patent Application Publication
(10) Pub. No.: US 2005/0201937 A1
(43) Pub. Date: Sep. 15, 2005

(54) Temporal or spatial characterization of biosynthetic events in living organisms by isotopic fingerprinting under conditions of imposed isotopic gradients

(75) Inventor: Marc K. Hellerstein, Kensington, CA (US)

Correspondence Address: MORRISON & FOERSTER LLP, 425 MARKET STREET, SAN FRANCISCO, CA, 94105-2482, US

(73) Assignee: The Regents of the University of California, Oakland, CA (US)

(21) Appl. No.: 11/078,083

(22) Filed: Mar. 11, 2005

Related U.S. Application Data
(60) Provisional application No. 60/552,675, filed on Mar. 11, 2004.

(51) Int. Cl. C12Q 1/00; C12Q 1/68; A61K 49/00; G06F 19/00; G01N 33/48; G01N 33/50; C07H 21/04

(52) U.S. Cl. 424/9.2; 435/4; 435/6; 530/350; 536/23.2; 702/19

(57) ABSTRACT

The invention provides methods useful for establishing timing or spatial location of a biosynthetic event in a living organism, without disrupting the event of interest and without disrupting the living organism. A temporal or spatial gradient of an isotopically labeled biochemical precursor is created, which serves to isotopically fingerprint (i.e., definitively mark) when or where biosynthesis occurs. The methods of the invention are broadly applicable to a variety of medical, public health, and diagnostic applications, as well as for establishing sequences of biochemical events that occur within a living organism.

### CROSS-REFERENCE TO RELATED APPLICATIONS

This application claims priority to U.S. provisional application No. 60/552,675 filed on Mar. 11, 2004 which is hereby incorporated by reference in its entirety.

### FIELD OF THE INVENTION

This invention relates to methods for determining temporal or spatial localization of a biosynthetic process of interest within a living organism. Upon creation of a temporal or spatial gradient of an isotopically-labeled biochemical precursor, label incorporated into a biochemical component of the living organism creates an isotopic fingerprint which may be used to establish timing or spatial location of the biosynthetic events.

### BACKGROUND OF THE INVENTION

Many biological processes have a temporal organization wherein a sequence of events is critical to the final outcome. Examples of temporally-organized biological processes include development, aging, growth, adaptation to environmental changes, sleep, formation of memory, and pathogenesis of most diseases (e.g., carcinogenesis, diabetogenesis, atherosclerosis, Alzheimer's progression, etc.). At a biochemical level, the cell cycle is timed with a resulting temporal pattern of DNA synthesis. The synthesis of other cellular macromolecules (e.g., proteins, lipids, complex carbohydrates) also exhibit distinctive temporal patterns.

Despite the importance of timing in biology, there has been no generally applicable noninvasive, post-hoc method to establish the timing or sequence of biochemical events in a living organism. Previous methods of establishing the timing of a biosynthetic event in vivo have required disruption of the process (e.g., by sampling the tissue or killing the experimental animal at timed intervals). Moreover, currently available methods for establishing the timing of biochemical processes must be performed in real time (i.e., tissue sampling at the time when each event is believed to occur), rather than after the fact, when the entire process has been completed. For complex processes that involve a long chain of biochemical events or where, for example, a molecule is synthesized at one site and subsequently migrates to another location, the requirement to sample each event at the precise time of its occurrence is a significant constraint. It would be preferable to be able to definitively mark, or “fingerprint,” a molecule at the time of its synthesis, and sample at a later time, when the entire biochemical process of interest has been completed.

Thus, there is a need for a method for establishing the timing (i.e., temporal localization) and spatial localization of biosynthetic events in a living organism that is noninvasive (i.e., does not require disruption of the system) and that can be applied ex post facto (i.e., after an entire process has been completed). Such a method would be of great utility in both biology and medicine, especially if it were broadly applicable to most classes of biomolecules.

### BRIEF SUMMARY OF THE INVENTION

In order to meet these needs, the present invention includes methods of determining the temporal or spatial location of biosynthetic processes in an organism.

Methods of determining the timing of biosynthesis involve administering one or more stable isotope-labeled biochemical precursors to an organism, and varying the amount administered over time to create a temporal gradient of isotopic enrichment in the precursor pool within the living organism. After the isotope labeled biochemical precursors are incorporated into one or more biochemical components of the living organism, one or more biological samples are obtained from the organism, and the isotopic labeling pattern within the biochemical components is measured. The observed isotopic labeling pattern of the biochemical component is compared to a predicted or theoretically-calculated isotopic labeling pattern to determine the timing of biosynthesis of the biochemical component. The measured isotopic fingerprint of a given biochemical component is dependent on the concentration of the isotope labeled precursor at the time said component was synthesized. The concentration of the isotope labeled precursor is what is varied over time to create the temporal gradient. Given this, comparison of the measured isotopic fingerprint with those predicted to occur across the range of concentration in the gradient allows for the determination of when on the gradient, and so when in time, synthesis occurred.

Administration of the isotope labeled biochemical precursor may be increased or decreased over time. If a plurality of biochemical precursors is administered, one precursor may be increased over time while another precursor may be decreased over time, for example, by use of combined stable isotope label administration protocols.

Methods of determining the spatial localization of biosynthesis involve administering a biochemical precursor comprising a detectable amount of an isotope label, and varying the amount of isotope label spatially within the organism to create a spatial gradient of isotopic enrichment (e.g., in one part of the brain more than in another part). After the isotope labeled biochemical precursors are incorporated into one or more biochemical components of the living organism, one or more biological samples is obtained from the organism, and the isotopic labeling pattern of the biochemical components is measured. The spatial localization of biosynthesis is then established by comparing the isotopic labeling pattern with predicted isotopic labeling patterns across the spatial gradient.

The labeling patterns of the biochemical components are compared to one another to establish their relative spatial location of biosynthesis.

Isotopic labels may include any stable isotope label found in biological systems. Examples of isotope labels include ²H, ¹³C, ¹⁵N, and ¹⁸O. In one embodiment, the isotope label is ²H, which may be administered in water (i.e., as ²H₂O).

The biochemical precursor may be any precursor known in the art. Examples of precursors include amino acids, monosaccharides, lipids, CO₂, NH₃, H₂O, nucleosides, and nucleotides.

Measured biochemical components include polypeptides, polynucleotides, purines, pyrimidines, amino acids, carbohydrates, lipids, and porphyrins.

The organism may be any known organism, including a prokaryotic cell, a eukaryotic cell, a mammal, or a human.

The biological sample may be collected at any time during or after the administration of the biochemical precursor. In one embodiment, the biological sample is collected at the termination of a biological process of interest.

The methods may be used to compare the timing of biosynthesis of different biochemical components of a complex physiologic mixture during biosynthesis. For example, the relative timing of lipid and amino acid synthesis in plasma lipoproteins may be determined.

The isotopic labeling pattern is determined by methods known in the art. For example, the isotopic labeling pattern may be determined by mass spectrometry. Alternatively, the isotopic labeling pattern may be determined by nuclear magnetic resonance (NMR) spectroscopy.

The present invention is further directed to a method of determining the timing of the synthesis of a biochemical component in a living organism. The method includes the following steps: a)administering one or more isotopically labeled biochemical precursors to an organism, wherein the amount of one or more isotopically labeled biochemical precursors administered are varied over time to create a temporal gradient of isotopic enrichment in a biochemical precursor pool within the living organism, and wherein the one or more isotopically labeled biochemical precursors are incorporated biosynthetically into one or more biochemical components of the living organism; b) obtaining one or more biological samples from the living organism, wherein the one or more biological samples includes one or more biochemical components; c) measuring the isotopic labeling pattern in the one or more biochemical components; and d) comparing the isotopic labeling pattern measured in step c) with a predicted isotopic labeling pattern across the temporal gradient or to another biochemical component in the living organism to determine the timing of biosynthesis of said biochemical component.

The present invention is further directed to a method for determining the spatial localization of a biosynthetic event in a living organism. The method may include the following steps: a) administering at least one biochemical precursor including a detectable amount of an isotopic label, wherein the amount of isotopic label administered varies spatially within the living organism to create a spatial gradient of isotopic enrichment in a biochemical precursor pool within the living organism, and wherein the at least one biochemical precursor is incorporated biosynthetically into one or more biochemical components of the living organism; b) isolating the one or more biochemical components from a biological sample of the living organism; c) determining the isotopic labeling pattern in the one or more biochemical components; and d) establishing the spatial location of biosynthesis of the one or more biochemical components by comparing the isotopic labeling pattern determined in step d) with predicted isotopic labeling patterns across the spatial gradient or to another biochemical component in the living organism.

In the method, the administering step a) may include increasing or decreasing the amount of the one or more isotopically labeled biochemical precursors over time.

In another format of the method, the administering step a) may include administering a plurality of isotopically labeled biochemical precursors, wherein the amount of at least one of the isotopically labeled biochemical precursors is increased over time and the amount of at least one of the isotopically labeled biochemical precursors is decreased over time.

In the method, the isotopic label may be chosen from ²H, ¹³C, ¹⁵N, and ¹⁸O. The biochemical precursor may be chosen from amino acids, monosaccharides, lipids, CO₂, NH₃, H₂O, nucleosides, and nucleotides. The biochemical component may be chosen from polypeptides, polynucleotides, purines, pyrimidines, amino acids, carbohydrates, lipids, and porphyrins.

In the method, the living organism may be a prokaryotic cell, a eukaryotic cell or a mammal. In one format, the mammal is a human.

In one format of the invention, the biological sample is collected at the termination of a biological process of interest. In another format, a plurality of biochemical components is isolated and the isotopic labeling patterns of the biochemical components are compared to one another to establish their relative timing of biosynthesis.

In the method, the isotopic labeling pattern may be determined by mass spectrometry or by NMR spectroscopy.

The invention is further directed to an information storage device including data obtained from the methods of the invention. In one format, the device is a printed report. The medium in which the report is printed on may be chosen from paper, plastic, and microfiche. In another format, the device is a computer disc. The disc may be chosen from a compact disc, a digital video disc, an optical disc, and a magnetic disc.

The present invention is further directed to an isotopically-perturbed molecule generated by the methods of the invention. The isotopically-perturbed molecule may be chosen from protein, lipid, nucleic acid, glycosaminoglycan, proteoglycan, porphyrin, and carbohydrate molecules.

### BRIEF DESCRIPTION OF THE DRAWINGS

FIG. 1 shows an increase in p values (derived from a comparison between predicted and actual labeling patterns across the gradient as calculated by MIDA) for bone marrow DNA (FIG. 1A), stromovascular retroperitoneal DNA (FIG. 1B), fat retroperitoneal DNA (FIG. 1C), and fat epithelial triglycerides (FIG. 1D). The observed increase in p values represents the influence of the temporal gradient on the isotopic fingerprint of the isolated DNA or triglyceride.

FIG. 2 depicts the consequences of an isotopic gradient in a biosynthetic precursor pool on the labeling pattern in polymeric products. A time gradient for ²H₂O is simulated here (from 0% to 6% body ²H₂O enrichment over a 21-day period). Mass isotopomer patterns in a protein-bound amino acid (alanine, n=4 hydrogen atoms from cellular H₂O), a triacylglycerol-bound fatty acid (palmitate, n=22 hydrogen atoms from H₂O), and a component of galactosyl-cerebroside (galactose, n=5) are shown. The mass isotopomer patterns differ for molecules synthesized from days 0-7 (left), 7-14 (middle), and 14-21 (right). Each pattern represents a permanent isotopic fingerprint of the time of synthesis. EMₓ, excess abundance in mass isotopomer M+x. Ratio, ratio EM₂/EM₁.

FIG. 3 diagrams the principle of combinatorial analysis (e.g., MIDA) depicting the biosynthetic precursor pool enrichment, the combinations of mass isotopomers, and calculated (predicted) mass isotopic labeling pattern. This figure represents the concept of combinatorial analysis that forms the basis of the MIDA calculation and allows one to predict the isotopic fingerprint of a biomolecule based on the value of p, or the reverse.

### DETAILED DESCRIPTION OF THE INVENTION

The invention provides methods and kits for determining the timing or spatial location of a biosynthetic event within a living organism. In the methods of the invention, a temporal or spatial gradient of an isotopic labeled biochemical precursor is created. Incorporation of the label into a biochemical component of the living organism creates an “isotopic fingerprint” which allows determination of when or where biosynthesis occurred by comparison with predicted labeling patterns across the gradient. Methods of the invention may be used to determine the timing of a biosynthetic event post hoc, in a living organism, without disrupting the ongoing process. Methods of the invention may also be used to observe or elucidate spatially organized processes in biology (i.e., gradients of synthesis across a tissue or organism).

Methods of the invention are useful for a variety of medical applications, for example, amniotic fluid diagnosis (i.e., to determine whether timed events have been disrupted in vivo, for example by exposure to a toxin). Methods of the invention may also be used for characterization of sequential events leading to development of a disease and for pharmaceutical and genetic research studies.

### ADVANTAGES OF THE INVENTION

(1) Creation of a gradient of isotope enrichment. Previous methods teach generation and maintenance of a relatively constant isotopic enrichment over time in the biosynthetic precursor pool in a cell or organism when used for the purpose of measuring biosynthetic rates. In contrast, the present invention teaches formation of a gradient of isotope enrichment in time or space, which allows determination of when or where a biosynthetic event takes place.

(2) Ability to measure a multiplicity of isotope enrichments simultaneously. Previous methods teach calculation of single, average isotope enrichment for a biosynthetic precursor pool in a cell or organism over the time period in which labeling occurs. In contrast, the present invention teaches a range of isotope enrichments for the biosynthetic pool (i.e., a temporal or spatial gradient), allowing differentiation of multiple (i.e., non-average) isotope enrichments in different molecules synthesized at different times or places.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology. A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (I. E. Coligan et al., eds., 1991); Short Protocols in Molecular Biology (Wiley and Sons, 1999); and Mass isotopomer distribution analysis at eight years: theoretical, analytic and experimental considerations by Hellerstein and Neese (Am J Physiol 276 (Endocrinol Metab. 39) E1146-E1162, 1999). Furthermore, procedures employing commercially available assay kits and reagents will typically be used according to manufacturer-defined protocols unless otherwise noted.

Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, Mass isotopomer distribution analysis at eight years: theoretical, analytic and experimental considerations by Hellerstein and Neese (Am J Physiol 276 (Endocrinol Metab. 39) E1146-E1162, 1999). As appropriate, procedures involving the use of commercially available kits and reagents are generally carried out in accordance with manufacturer defined protocols and/or parameters unless otherwise noted.

“Isotopes” or “mass isotopic atoms” refers to atoms with the same number of protons and hence of the same element but with different numbers of neutrons (e.g., H vs. ²H, or D). Examples of isotopes suitable for use as isotopic labels include, but are not limited to, ²H, ¹³C, ¹⁵N, ¹⁷O, and ¹⁸O.

An “isotopic label” or “isotope label” refers to a detectable amount of a mass isotopic atom, incorporated into the molecular structure of the biochemical precursor to be administered. In one embodiment, the label is “stable,” or does not decay with release of energy but persists in a stable manner.

“Mass isotopomers” of a molecule are identical chemical structures which differ only in mass to charge ratio, or roughly, molecular weight, due to the presence of one or more selected mass isotopic atoms.

An “isotope-labeled biochemical precursor” refers to any molecule that contains an isotope of an element at levels above that found in natural abundance molecules.

A “biochemical component” is a molecule of a living organism which is synthesized from one or more biochemical precursors. Often, a biochemical component is a “biopolymer” or “macromolecule,” a molecule that is synthesized in a biological system using discrete subunits as precursors.

“Labeled water” includes water labeled with one or more specific heavy isotopes of either hydrogen or oxygen. Specific examples of labeled water include ²H₂O and H₂¹⁸O.

“Partially purifying” refers to methods of removing one or more components of a mixture of other similar compounds. For example, “partially purifying a protein or peptide” refers to removing one or more proteins or peptides from a mixture of one or more proteins or peptides.

“Isolating” refers to separating one compound from a mixture of compounds. For example, “isolating a protein or peptide” refers to separating one specific protein or peptide from all other proteins or peptides in a mixture of one or more proteins or peptides.

As used herein, a “living organism” is an organism which incorporates a biochemical precursor molecule into a macromolecule via biosynthesis. A living organism may be prokaryotic, eukaryotic, or viral. A living organism may be single-celled or multicellular. Often, a living organism is a vertebrate, typically a mammal. The term “mammal” includes humans, nonhuman primates, farm animals, pet animals, for example cats and dogs, and research animals, for example mice and rats. In some embodiments, the living organism is a tissue culture cell, for example, of mammalian, insect, or plant origin.

A “detectable amount” of an isotopic label is an amount that can be measured after incorporation into a biochemical component of a living organism, using any method suitable for quantitation of such isotopes. Examples of these methods include mass spectrometry, nuclear magnetic resonance (NMR) spectroscopy, chemical fragmentation, liquid scintillation, and other methods known in the art.

By “predicted isotopic labeling pattern” is meant the quantitative distribution of the stable isotopic label into different mass isotopomers that is predicted or calculated from combinatorial analysis, by hand, or by algorithm (details discussed, infra).

By “isotopic fingerprint” is meant the quantitative distribution or pattern of the isotopic label into different mass isotopomers in a biochemical component, either as predicted (from combinatorial analysis, by hand, or by algorithm) or measured (details discussed, infra).

Methods

Methods of determining the timing and spatial localization of a biosynthetic event are disclosed herein. In one embodiment of the invention, an isotope-labeled biochemical precursor is administered to a living organism by varying the amount of label administered over time. One or more biological samples are obtained from the organism and the isotope labeling pattern of one or more biological components are compared to a predicted isotopic pattern across a temporal gradient to determine the timing of biosynthesis of the biological component. The predicted or calculated isotopic pattern is calculated using the MIDA equations (combinatorial analysis) or analogous calculation approaches known in the art appropriate for the biological component being analyzed. The isotopic pattern predicted or calculated by these equations is dependent on the concentration or enrichment of the isotope labeled precursor, and this concentration is what is increased or decreased over time to create the temporal gradient. The comparison of the measured isotopic distribution to that predicted, for example by the MIDA calculations, allows for the determination of the concentration of isotope-labeled precursor at the time of synthesis of the biological component being analyzed. The concentration of the isotope labeled precursor at any given time is known, based on the protocol for its administration, measurements made from biological samples taken during the period of label administration, or previous similar experiments. Comparing the measured isotopic distribution to predicted or calculated isotopic distributions allows for the determination of the concentration of label at the time of synthesis in the biosynthetic precursor pool for a biochemical component, which in turn allows for the determination of the time that the synthesis occurred.

In another embodiment of the invention, a stable isotope-labeled biochemical precursor is administered to a living organism by spatially varying the amount of label administered. One or more biological samples are obtained from the organism and the isotope labeling pattern of one or more biological components are compared to a predicted isotopic pattern across a spatial gradient to determine the location of biosynthesis of the biological component or components. The predicted isotopic pattern is calculated using the MIDA equations (combinatorial analysis) or analogous calculation approaches known in the art appropriate for the biological component being analyzed. The isotopic pattern predicted by these equations is dependent on the concentration of the isotope labeled precursor, and this concentration or enrichment is what varies between different compartments of the living system in question, in order to create the spatial labeling gradient. The comparison of the measured isotopic distribution to that predicted, for example, by the MIDA calculations allows for the determination of the concentration of isotope-labeled precursor in the compartment where the synthesis of the biological component being analyzed occurred. The concentration of the isotope labeled precursor in different compartments is known, based on the protocol for its administration, measurements made from biological samples taken during the period of label administration, or previous similar experiments. Comparing the measured isotopic distribution to predicted isotopic distributions allows for the determination of the concentration of label in the compartment where synthesis occurred, which in turn allows for the determination of the place or compartment where the synthesis occurred.

A. Administering one or more Isotope-Labeled Biochemical Precursors

1. Isotope-Labeled Biochemical Precursors

a. Isotope Labels

The first step in determining the timing of or spatial localization of a biochemical event involves administering one or more isotope-labeled biochemical precursors to a living organism. Stable isotope labels that can be used include, but are not limited to, ²H, ¹³C, ¹⁵N, ¹⁸O or other stable isotopes of elements present in organic systems.

In one embodiment, the isotope label is ²H.

b. Biochemical Precursors

A labeled biochemical precursor must be capable of metabolic entry into the nutrient metabolic pools of the living organism. In methods of the invention, a biochemical component of the living organism becomes isotopically labeled via biosynthesis, incorporating one or more isotope labeled biochemical precursors from the precursor pool into the component.

The biochemical precursor molecule may be any molecule that is metabolized in the body to form a biological molecule. Isotope labels may be used to modify all biochemical precursor molecules disclosed herein, and indeed all biochemical precursor molecules, to form isotope-labeled biochemical precursor molecules.

The entire biochemical precursor molecule may be incorporated into one or more biological molecules. Alternatively, a portion of the biochemical precursor molecule may be incorporated into one or more biological molecules.

Biochemical precursor molecules may include, but are not limited to, CO₂, NH₃, glucose (and other sugars), amino acids, triglycerides, lactate, H₂O, acetate, and fatty acids.

i. Water as a Biochemical Precursor Molecule

Water is a biochemical precursor of proteins, polynucleotides, lipids, carbohydrates, modifications or combinations thereof, and other biological molecules. As such, labeled water (e.g., ²H₂O) may serve as a biochemical precursor in the methods taught herein.

Labeled water may be readily obtained commercially. For example, ²H₂O may be purchased from Cambridge Isotope Labs (Andover, Mass.).

Labeled water may be used as a near-universal biochemical precursor for most classes of biological molecules.

ii. Protein, Oligonucleotide, Lipid, and Carbohydrate Biochemical Precursors

Examples of biochemical precursor molecules include biochemical precursors of proteins, polynucleotides, lipids, and carbohydrates.

### Biochemical Precursors of Proteins

The biochemical precursor molecule may be any biochemical precursor molecule for protein synthesis known in the art. These biochemical precursor molecules may include, but are not limited to, CO₂, NH₃, glucose, lactate, H₂O, acetate, and fatty acids.

Biochemical precursor molecules of proteins may also include one or more amino acids. The biochemical precursor may be any amino acid. The biochemical precursor molecule may be a singly or multiply deuterated amino acid. The biochemical precursor molecule may be one or more of ¹³C-lysine, ¹⁵N-histidine, ¹³C-serine, ¹³C-glycine, ²H-leucine, ¹⁵N-glycine, ¹³C-leucine, ²H₅-histidine, and any deuterated amino acid. Labeled amino acids may be administered, for example, undiluted with non-deuterated amino acids. All isotope labeled biochemical precursors may be purchased commercially, for example, from Cambridge Isotope Labs (Andover, Mass.).

The biochemical precursor molecule may also include any biochemical precursor for post-translational or pre-translationally modified amino acids. These biochemical precursors may include, but are not limited to, precursors of methylation such as glycine, serine or H₂O; precursors of hydroxylation, such as H₂O or O₂; precursors of phosphorylation, such as phosphate, H₂O or O₂; precursors of prenylation, such as fatty acids, acetate, H₂O, ethanol, ketone bodies, glucose, or fructose; precursors of carboxylation, such as CO₂, O₂, H₂O, or glucose; precursors of acetylation, such as acetate, ethanol, glucose, fructose, lactate, alanine, H₂O, CO₂, or O₂; and other post-translational modifications known in the art.

The degree of labeling present in free amino acids may be determined experimentally, or may be assumed based on the number of labeling sites in an amino acid. For example, when using hydrogen isotopes as a label, the labeling present in C—H bonds of free amino acids or, more specifically, in tRNA-amino acids, during exposure to ²H₂O in body water may be identified. The total number of C—H bonds in each non-essential amino acid is known—e.g., 4 in alanine, 2 in glycine.

The biochemical precursor molecule for proteins may be water. The hydrogen atoms on C—H bonds are the hydrogen atoms on amino acids that are useful for measuring protein synthesis from ²H₂O since the O—H and N—H bonds of peptides and proteins are labile in aqueous solution. As such, the exchange of ²H-label from ²H₂O into O—H or N—H bonds occurs without the synthesis of proteins from free amino acids as described above. C—H bonds undergo incorporation from H₂O into free amino acids during specific enzyme-catalyzed intermediary metabolic reactions. The presence of ²H-label in C—H bonds of protein-bound amino acids after ²H₂O administration therefore means that the protein was assembled from amino acids that were in the free form during the period of ²H₂O exposure—i.e., that the protein is newly synthesized. Analytically, the amino acid derivative used must contain all the C—H bonds but must remove all potentially contaminating N—H and O—H bonds.

Hydrogen atoms from body water may be incorporated into free amino acids. ²H from labeled water can enter into free amino acids in the cell through the reactions of intermediary metabolism, but ²H cannot enter into amino acids that are present in peptide bonds or that are bound to transfer RNA. Free essential amino acids may incorporate a single hydrogen atom from body water into the α-carbon C—H bond, through rapidly reversible transamination reactions. Free non-essential amino acids contain a larger number of metabolically exchangeable C—H bonds, of course, and are therefore expected to exhibit higher isotopic enrichment values per molecule from ²H₂O in newly synthesized proteins.

One of skill in the art will recognize that labeled hydrogen atoms from body water may be incorporated into other amino acids via other biochemical pathways. For example, it is known in the art that hydrogen atoms from water may be incorporated into glutamate via synthesis of the biochemical precursor α-ketoglutarate in the citric acid cycle. Glutamate, in turn, is known to be the biochemical precursor for glutamine, proline, and arginine. By way of another example, hydrogen atoms from body water may be incorporated into post-translationally modified amino acids, such as the methyl group in 3-methyl-histidine, the hydroxyl group in hydroxyproline or hydroxylysine, and others. Other amino acids synthesis pathways are known to those of skill in the art.

Oxygen atoms (H₂¹⁸O) may also be incorporated into amino acids through enzyme-catalyzed reactions. For example, oxygen exchange into the carboxylic acid moiety of amino acids may occur during enzyme catalyzed reactions. Incorporation of labeled oxygen into amino acids is known to one of skill in the art. Oxygen atoms may also be incorporated into amino acids from 18O₂ through enzyme catalyzed reactions (including hydroxyproline, hydroxylysine or other post-translationally modified amino acids).

Hydrogen and oxygen labels from labeled water may also be incorporated into amino acids through post-translational modifications. In one embodiment, the post-translational modification may already include labeled hydrogen or oxygen through biosynthetic pathways prior to post-translational modification. In another embodiment, the post-translational modification may incorporate labeled hydrogen, oxygen, carbon, or nitrogen from metabolic derivatives involved in the free exchange labeled hydrogens from body water, either before or after a post-translational modification step (e.g., methylation, hydroxylation, phosphorylation, prenylation, sulfation, carboxylation, acetylation or other known post-translational modifications).

### Biochemical Precursors of Polynucleotides

The biochemical precursor molecule may include components of polynucleotides. Polynucleotides include purine and pyrimidine bases and a ribose-phosphate backbone. The biochemical precursor molecule may be any polynucleotide biochemical precursor molecule known in the art.

The biochemical precursor molecules of polynucleotides may include, but are not limited to, CO₂, NH₃, urea, O₂, glucose, lactate, H₂O, acetate, ketone bodies and fatty acids, glycine, succinate or other amino acids, and phosphate.

Biochemical precursor molecules of polynucleotides may also include one or more nucleoside residues. The biochemical precursor molecules may also be one or more components of nucleoside residues. Glycine, aspartate, glutamine, and tetrahydrofolate, for example, may be used as biochemical precursor molecules of purine rings. Carbamyl phosphate and aspartate, for example, may be used as biochemical precursor molecules of pyrimidine rings. Adenine, adenosine, guanine, guanosine, cytidine, cytosine, thymine, or thymidine may be given as biochemical precursor molecules for deoxyribonucleosides. All isotope labeled biochemical precursors may be purchased commercially, for example, from Cambridge Isotope Labs (Andover, Mass.).

The biochemical precursor molecule of polynucleotides may be water. The hydrogen atoms on C—H bonds of polynucleotides, polynucleosides, and nucleotide or nucleoside precursors may be used to measure polynucleotide synthesis from ²H₂O. C—H bonds undergo exchange from H₂O into polynucleotide precursors. The presence of ²H-label in C—H bonds of polynucleotides, nucleosides, and nucleotide or nucleoside precursors, after ²H₂O administration therefore means that the polynucleotide was synthesized during this period. The degree of labeling present may be determined experimentally, or assumed based on the number of labeling sites in a polynucleotide or nucleoside.

Hydrogen atoms from body water may be incorporated into free nucleosides or polynucleotides. ²H from labeled water can enter these molecules through the reactions of intermediary metabolism.

One of skill in the art will recognize that labeled hydrogen atoms from body water may be incorporated into other polynucleotides, nucleotides, or nucleosides via various biochemical pathways. For example, glycine, aspartate, glutamine, and tetrahydrofolate, which are known biochemical precursor molecules of purine rings. Carbamyl phosphate and aspartate, for example, are known biochemical precursor molecules of pyrimidine rings. Ribose and ribose phosphate, and their synthesis pathways, are known biochemical precursors of polynucleotide synthesis.

Oxygen atoms (H₂¹⁸O) may also be incorporated into polynucleotides, nucleotides, or nucleosides through enzyme-catalyzed biochemical reactions, including those listed above. Oxygen atoms from 18O₂ may also be incorporated into nucleotides by oxidative reactions, including non-enzymatic oxidation reactions (including oxidative damage, such as formation of 8-oxo-guanine and other oxidized bases or nucleotides).

Isotope-labeled biochemical precursors may also be incorporated into polynucleotides, nucleotides, or nucleosides in post-replication modifications. Post-replication modifications include modifications that occur after synthesis of DNA molecules. The metabolic derivatives may be methylated bases, including, but not limited to, methylated cytosine. The metabolic derivatives may also be oxidatively modified bases, including, but not limited to, 8-oxo-guanosine. Those of skill in the art will readily appreciate that the label may be incorporated during synthesis of the modification.

### Biochemical Precursors of Lipids

Labeled biochemical precursors of lipids may include any precursor in lipid biosynthesis.

The biochemical precursor molecules of lipids may include, but are not limited to, CO₂, NH₃, glucose, lactate, H₂O, acetate, and fatty acids.

The biochemical precursor may also include labeled water, for example ²H₂O, which is a biochemical precursor of fatty acids, the glycerol moiety of acyl-glycerols, cholesterol and its derivatives; ¹³C or ²H-labeled fatty acids, which are biochemical precursors of triglycerides, phospholipids, cholesterol ester, coamides and other lipids; ¹³C— or ²H-acetate, which is a biochemical precursor of fatty acids and cholesterol; ¹⁸O₂, which is a biochemical precursor of fatty acids, cholesterol, acyl-glycerides, and certain oxidatively modified fatty acids (such as peroxides) by either enzymatically catalyzed reactions or by non-enzymatic oxidative damage (e.g., to fatty acids); ¹³C- or ²H-glycerol, which is a biochemical precursor of acyl-glycerides; ¹³C- or ²H-labeled acetate, ethanol, ketone bodies or fatty acids, which are biochemical precursors of endogenously synthesized fatty acids, cholesterol and acylglycerides; and ²H or ¹³C-labeled cholesterol or its derivatives (including bile acids and steroid hormones). All isotope labeled biochemical precursors may be purchased commercially, for example, from Cambridge Isotope Labs (Andover, Mass.).

Complex lipids, such as glycolipids and cerebrosides, can also be labeled from biochemical precursors, including ²H₂O, which is a biochemical precursor of the sugar-moiety of cerebrosides (including, but not limited to, N-acetylgalactosamine, N-acetylglucosamine-sulfate, glucuronic acid, and glucuronic acid-sulfate), the fatty acyl-moiety of cerebrosides and the sphingosine moiety of cerebrosides; ²H- or ¹³C-labeled fatty acids, which are biochemical precursors of the fatty acyl moiety of cerebrosides, glycolipids and other derivatives.

The biochemical precursor molecule may be or include components of lipids.

### Biochemical precursors of Glycosaminoglycans and Proteoglycans

Glycosaminoglycans and proteoglycans are a complex class of biomolecules that play important roles in the extracellular space (e.g., cartilage, ground substance, and synovial joint fluid). Molecules in these classes include, for example, the large polymers built from glycosaminoglycan disaccharides, such as hyaluronan, which is a polymer composed of up to 50,000 repeating units of hyaluronic acid (HA) disaccharide, a dimer that contains N-acetyl-glucosamine linked to glucuronic acid; chondroitin-sulfate (CS) polymers, which are built from repeating units of CS disaccharide, a dimer that contains N-acetyl-galactosamine-sulfate linked to glucuronic acid, heparan-sulfate polymers, which are built from repeating units of heparan-sulfate, a dimer of N-acetyl (or N-sulfo)-glucosamine-sulfate linked to glucuronic acid; and keratan-sulfate polymers, which are built from repeating units of keratan-sulfate disaccharide, a dimer that contains N-acetylglucosamine-sulfate liked to galactose. Proteoglycans contain additional proteins that are bound to a central hyaluronan polymer and other glycosaminoglycans, such as CS, that branch off of the central hyaluronan chain.

Labeled biochemical precursors of glycosaminoglycans and proteoglycans include, but are not limited to, ²H₂O (incorporated into the sugar moieties, including N-acetylglucosamine, N-acetylgalactosamine, glucuronic acid, the various sulfates of N-acetylglucosamine and N-acetylgalactosamine, galactose, iduronic acid, and others), ¹³C- or ²H-glucose (incorporated into said sugar moieties), ²H- or ¹³C-fructose (incorporated into said sugar moieties), ²H- or ¹³C-galactose (incorporated into said sugar moieties), ¹⁵N-glycine, other ¹⁵N-labeled amino acids, or ¹⁵N-urea (incorporated into the nitrogen-moiety of said amino sugars, such as N-acetylglucosamine, N-acetyl-galactosamine, etc.); ¹³C- or ²H-fatty acids, ¹³C- or ²H-ketone bodies, ¹³C-glucose, ¹³C-fructose, ¹⁸O₂, ¹³C- or ²H-acetate (incorporated into the acetyl moiety of N-acetyl-sugars, such as N-acetyl-glucosamine or N-acetyl-galactosamine), and ¹⁸O-labeled sulfate (incorporated into the sulfate moiety of chondroitin-sulfate, heparan-sulfate, keratan-sulfate, and other sulfate moieties). All isotope labeled biochemical precursors may be purchased commercially, for example, from Cambridge Isotope Labs (Andover, Mass.).

### Biochemical Precursors of Carbohydrates

Labeled biochemical precursors of carbohydrates may include any biochemical precursor of carbohydrate biosynthesis known in the art. These biochemical precursor molecules include but are not limited to H₂O, monosaccharides (including glucose, galactose, mannose, fucose, glucuronic acid, glucosamine and its derivatives, galactosamine and its derivatives, iduronic acid, fructose, ribose, deoxyribose, sialic acid, erythrose, sorbitol, adols, and polyols), fatty acids, acetate, ketone bodies, ethanol, lactate, alanine, serine, glutamine and other glucogenic amino acids, glycerol, O₂, CO₂, urea, starches, disaccharides (including sucrose, lactose, and others), glucose polymers and other polymers of said monosaccharides (including complex polysaccharides).

The biochemical precursor molecule may include labeled water, for example ²H₂O, which is a biochemical precursor to monosaccharides, ¹³C-labeled glucogenic biochemical precursors (including glycerol, CO₂, glucogenic amino acids, lactate, ethanol, acetate, ketone bodies and fatty acids), ¹³C- or ²H-labeled monosaccharides, ¹³C- or ²H-labeled starches or disaccharides; other components of carbohydrates labeled with ²H or ¹³C; and ¹⁸O₂, which is a biochemical precursor to monosaccharides and complex polysaccharides.

2. Methods of Administering Labeled Biochemical Precursor Molecules

Administration of an isotopically-labeled biochemical precursor to a host organism may be accomplished by a variety of methods that are well known in the art including oral, parenteral, subcutaneous, intravascular (e.g., intravenous and intraarterial), intraperitoneal, intramuscular, intranasal, and intrathecal administration. The delivery may be systemic, regional, or local. The biochemical precursor may be administered to a cell, a tissue, or systemically to a whole organism. The biochemical precursor may be formulated into appropriate forms for different routes of administration as described in the art, for example, in “Remington: The Science and Practice of Pharmacy,” Mack Publishing Company, Pennsylvania, 1995.

The labeled biochemical precursor may be provided in a variety of formulations, including solutions, emulsions, suspensions, powder, tablets, and gels, and/or may be optionally incorporated in a controlled-release matrix. The formulations may include excipients available in the art, such as diluents, solvents, buffers, solubilizers, suspending agents, viscosity controlling agents, binders, lubricants, surfactants, preservatives, and stabilizers. The formulations may include bulking agents, chelating agents, and antioxidants. Where parenteral formulations are used, the formulation may additionally or alternately include sugars, amino acids, or electrolytes.

Creation of a Temporal Gradient

In one embodiment, one or more isotopically labeled biochemical precursors is administered as described above in an amount that varies over time to create a temporal gradient of isotopic enrichment in the precursor pool within the living organism, or a cell or tissue thereof. A temporal gradient may be created either by increasing or decreasing the amount of an isotopically labeled precursor over time.

The isotopic enrichment in a biochemical precursor pool may be increased by methods that are well known in the art. For example, the isotopically labeled biochemical precursor may be repeatedly administered, administered in escalating doses, administered in doses that increase in frequency over time, or coadministered with agents that slow removal or accelerate uptake, or administered incorporated into a controlled or sustained-release matrix from which release accelerates over time, such as, for example, an implantable bioerodible polymeric matrix.

Alternatively, the isotopic enrichment of a labeled biochemical precursor may be decreased over time by methods known in the art such as, for example, diminishing doses, less frequent doses, a single initial dose, or coadministration of agents that speed removal or slow uptake.

In some embodiments, one or more labeled biochemical precursors are added in increasing amounts and one or more labeled biochemical precursors are added in decreasing amounts during overlapping or sequential time frames. Such increasing and decreasing gradients may be initiated simultaneously or may be started at different time points.

Creation of a Spatial Gradient

In some embodiments of the invention, one or more isotopically labeled biochemical precursors are administered such that a spatial gradient of isotopic enrichment is created in the precursor pool within the living organism, or tissue thereof. For example, a labeled biochemical precursor may be administered to a selected site within the living organism or within a tissue of the organism. A spatial gradient is created by diffusion or transport of the biochemical precursor away from the site of administration, or by differential administration of the isotopically labeled biochemical precursor across the physical space of a tissue or whole organism.

Obtaining One or more Biological Samples Comprising One or more Labeled Biochemical Components

After administration of a labeled biochemical precursor to a living organism and creation of a temporal or spatial gradient of isotope enrichment, one or more biochemical components are isolated from the living organism. When the living organism is a higher organism, such as a mammal, the biochemical component is isolated from a tissue or bodily fluid. Samples may be collected at a single time point or at multiple time points from one or more tissues or bodily fluids and/or at multiple locations within the living organism or a tissue thereof. The tissue or fluid may be collected using standard techniques in the art, such as, for example, tissue biopsy, blood draw, or collection of secretia or excretia from the body. Entire tissues, entire organs, or entire living systems may be collected. Examples of suitable bodily fluids or tissues from which a biochemical component may be isolated include, but are not limited to, urine, blood, intestinal fluid, edema fluid, saliva, lacrimal fluid (tears), cerebrospinal fluid, pleural effusions, sweat, pulmonary secretions, seminal fluid, feces, bile, intestinal secretions, or any suitable tissue in which a biochemical component of interest is synthesized or stored.

Samples may be collected at the termination of a biochemical process of interest, or at one or more time points intermediate between administration and termination of the biochemical process. Samples may be collected from a single location or from a plurality of locations. In some embodiments of the invention, both a temporal gradient and a spatial gradient may be created. In these embodiments, it may be desirable to collect samples at multiple time points (temporal gradient) and at multiple locations (spatial gradient).

The one or more biochemical components may also be purified, partially purified, or optionally, isolated, by conventional purification methods including, but not limited to, high performance liquid chromatography (HPLC), fast performance liquid chromatography (FPLC), chemical extraction, thin layer chromatography, gas chromatography, gel electrophoresis, and/or other separation methods known to those skilled in the art.

In another embodiment, the one or more biochemical components may be hydrolyzed or otherwise degraded to form smaller molecules. Hydrolysis methods include any method known in the art, including, but not limited to, chemical hydrolysis (such as acid hydrolysis) and biochemical hydrolysis (such as peptidase or nuclease degradation). Hydrolysis or degradation may be conducted either before or after purification and/or isolation of the biochemical component. The biochemical components also may be partially purified, or optionally, isolated, by conventional purification methods including, but not limited to, HPLC, FPLC, gas chromatography, gel electrophoresis, and/or any other methods of separating chemical and/or biochemical compounds known to those skilled in the art.

Determination of Isotopic Fingerprint

The “isotopic fingerprint” or “isotopomeric fingerprint” (i.e., isotopic labeling pattern) of biochemical components may be determined by methods known in the art. Such methods include, but are not limited to, mass spectrometry and NMR spectroscopy.

Isotopic enrichment in biochemical components can be determined by various methods such as mass spectrometry, including, but not limited to, gas chromatography-mass spectrometry (GC-MS), liquid chromatography-MS, electrospray ionization-MS, matrix assisted laser desorption-time of flight-MS, and Fourier-transform-ion-cyclotron-resonance-MS, cycloidal-MS.

Incorporation of labeled isotopes into biochemical components may be measured directly. Alternatively, incorporation of labeled isotopes may be determined by measuring the incorporation of labeled isotopes into one or more biochemical components, or hydrolysis or degradation products of biochemical components. The hydrolysis products may optionally be measured following either partial purification or isolation by any known separation method, as described previously.

a. Mass Spectrometry

Mass spectrometers convert components of a sample into rapidly moving gaseous ions and separate them on the basis of their mass-to-charge ratios. The distributions of isotopes or isotopologues of ions, or ion fragments, may thus be used to measure the isotopic enrichment in one or more metabolic derivatives.

Generally, mass spectrometers include an ionization means and a mass analyzer. A number of different types of mass analyzers are known in the art. These include, but are not limited to, magnetic sector analyzers, electrostatic analyzers, quadrupoles, ion traps, time of flight mass analyzers, and fourier transform analyzers. In addition, two or more mass analyzers may be coupled (MS/MS) first to separate precursor ions, then to separate and measure gas phase fragment ions.

Mass spectrometers may also include a number of different ionization methods. These include, but are not limited to, gas phase ionization sources such as electron impact, chemical ionization, and field ionization, as well as desorption sources, such as field desorption, fast atom bombardment, matrix assisted laser desorption/ionization, and surface enhanced laser desorption/ionization.

In addition, mass spectrometers may be coupled to separation means such as gas chromatography (GC) and HPLC. In gas-chromatography mass-spectrometry (GC/MS), capillary columns from a gas chromatograph are coupled directly to the mass spectrometer, optionally using a jet separator. In such an application, the GC column separates sample components from the sample gas mixture and the separated components are ionized and chemically analyzed in the mass spectrometer.

When GC/MS is used to measure mass isotopomer abundances of organic molecules, hydrogen-labeled isotope incorporation from labeled water is amplified 3 to 7-fold, depending on the number of hydrogen atoms incorporated into the organic molecule from labeled water.

In one embodiment, isotope enrichments of biochemical components may be measured directly by mass spectrometry.

In another embodiment, the biochemical components may be partially purified, or optionally isolated, prior to mass spectral analysis. Furthermore, hydrolysis or degradation products of metabolic derivatives may be purified.

In another embodiment, isotope enrichments of biochemical components after hydrolysis are measured by gas chromatography-mass spectrometry.

In an exemplary embodiment, the isotopic fingerprint is measured by quantitative mass spectrometry. This technique includes (a) measurement of relative abundances of different mass isotopomers (i.e., “isotope ratios”), (b) mass spectrometric fragmentation of molecules of interest and analysis of the fragments for relative abundances of different mass isotopomers, or (c) chemical or biochemical cleavage or rearrangement of molecules of interest prior to mass spectrometric measurement by the techniques of (a) or (b).

Establishing of Timing or Spatial Location of Biosynthesis

The observed isotopic fingerprint, measured as described above, is compared to predicted isotopic fingerprints. For the entire possible range of isotope precursor concentration (i.e., for the entire extent of the gradient) the predicted isotopic fingerprints are calculated according to equations known in the art (e.g., MIDA, combinatorial analysis). The measured isotopic fingerprint is compared to the predicted range of isotopic fingerprints, and the point at which it matches most closely represents the point on the gradient at which synthesis occurs. Alternatively, the measured isotopic fingerprints are compared in different biochemical compounds isolated, or in compounds isolated from different spatial locations. The equations used to predict the isotopic fingerprint describe the relationship between the concentration of the isotope-labeled precursor (which varies across the gradient) and the isotopic fingerprint of a biomolecule that is synthesized in the presence of that precursor. The equations allow for the calculation of predicted isotopic fingerprints from a known or assumed concentration of isotope-labeled precursor. The equations also allow for the calculation of the isotopic concentration in the isotope-labeled precursor pool from a measured isotopic fingerprint. The convergence of the predicted and measured values will occur at a concentration of isotope-labeled precursor that represents the value at the time or place of synthesis. This point is then located in the temporal or spatial gradient, and used to pinpoint the time or place of synthesis. The gradient is known, either from historical data, direct or indirect measurement previous to and during the labeling period. The isotopic concentration in the isotope-labeled precursor pool is sometimes referred to as “p”.

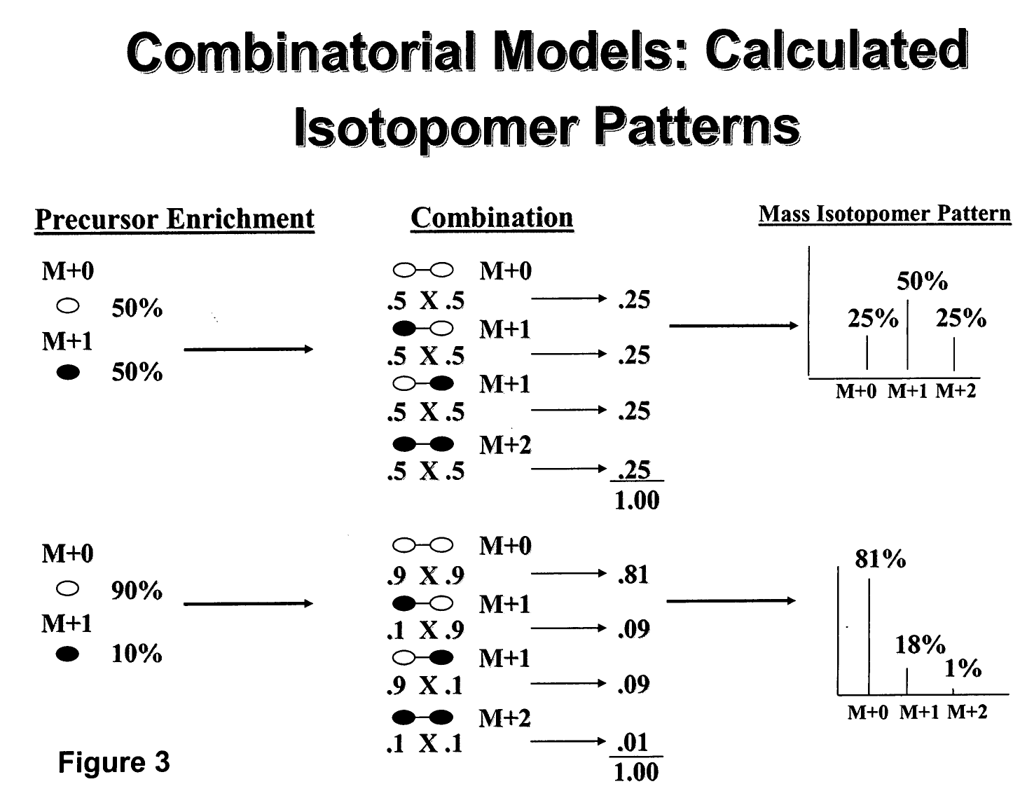

The age or location for a molecule based on where on the isotopic temporal or spatial gradient it may be found may be calculated by combinatorial analysis, by hand or via an algorithm. Variations of Mass Isotopomer Distribution Analysis (MIDA) combinatorial algorithm are discussed in a number of different sources known to one skilled in the art. Specifically, the MIDA calculation methods are the subject of U.S. Pat. No. 5,336,686, incorporated herein by reference. The method is further discussed by Hellerstein and Neese (1999), as well as Chinkes, et al. (1996), and Kelleher and Masterson (1992), all of which are hereby incorporated by reference in their entirety and is shown graphically in FIG. 3.

In addition to the above-cited references, calculation software implementing the method is publicly available from Professor Marc Hellerstein, University of California, Berkeley.

The biochemical component may be any biochemical component in the organism. Biochemical components include proteins, polynucleotides, fats, carbohydrates, porphyrins, and the like.

The methods disclosed herein may be used to determine the timing of biochemical synthesis during the development of an organism. For example, the timing of fat biosynthesis in developing mouse fetuses may be determined as in Example 1, infra.

The methods disclosed herein may also be used to determine the timing of biochemical components in humans. For example, blood samples taken in human subjects may be used to determine the timing of plasma protein and triglyceride synthesis in human lipoproteins as in Example 2, infra. For example, by decreasing the amount of body water in human subjects over time, the timing of ²H incorporation in amino acids of lipoproteins may be determined and compared to the timing of ²H incorporation in lipids.

The methods disclosed herein may also be used to identify the timing of organ generation. For example, the timing of pancreatic islet generation in a mammal may be determined.

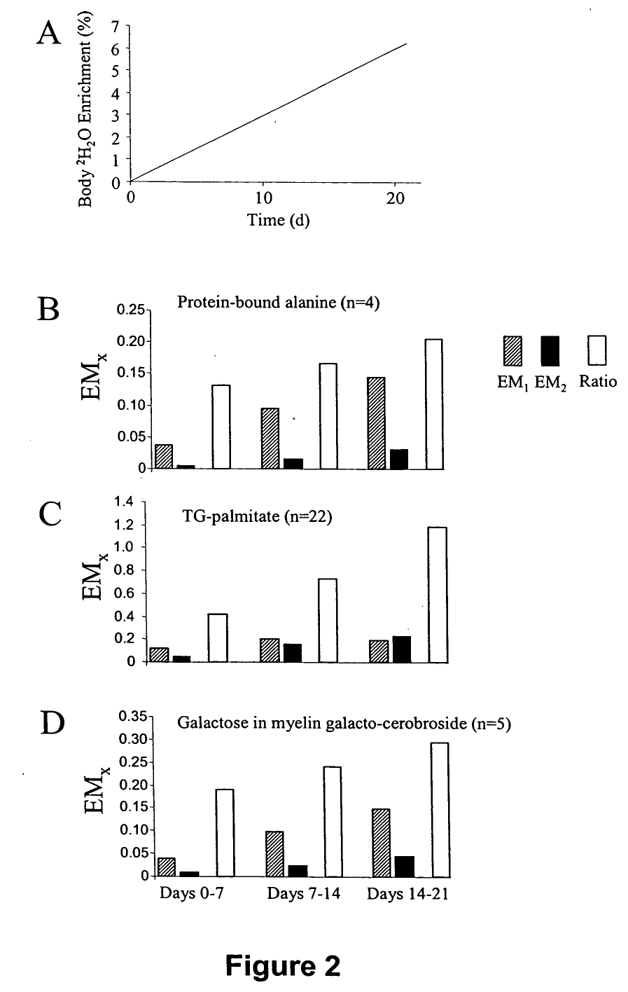

The timing of biosynthetic events in an organism can be established, post-hoc, by use of combinatorial probabilities (e.g., by use of MIDA, discussed supra). This is because the mass isotopomer pattern generated in a population of newly synthesized polymers retains its “isotopomeric fingerprint” throughout its lifespan. If an isotopic gradient is imposed over time, the isotopomeric fingerprint thereby reveals the time of synthesis, post-hoc, without having to stop the experiment (i.e., kill the animal). For example, if a pregnant dam is exposed to increased ²H₂O enrichments in drinking water (see FIG. 2), and lipids or protein are isolated from a portion of brain or some other tissue after birth of the fetus, the isotopomeric pattern will reveal the developmental time period during which the molecule was synthesized in the fetus.

In some embodiments, a plurality of biochemical components is isolated and the isotopic labeling patterns of each component are compared to one another to establish their relative timing or spatial location of biosynthesis.

The methods herein have several clinical applications. For example, the methods may be used to identify the timing or location of drug activity in an organism, which finds use in providing pharmacokinetic and pharmacodynamic information. The methods may also be used to determine whether an organism has a disease at one or more times by monitoring the timing of, for example, an immune response or other characteristic of a disease, which finds use in medical diagnoses and prognoses.

The methods herein have several public health applications. For example, the methods may be used to determine where an organism develops an adverse response to an exogenous chemical (i.e., xenobiotic agent) from, for example, exposure to one or more food additives, one or more industrial or occupational chemicals, or one or more environmental pollutants. The methods may be used to determine when an organism generates an adverse response to an exogenous chemical (i.e., in relation to time of exposure) and in what tissue or organism the response is located.

Kits

Kits for carrying out the methods disclosed herein are disclosed. Kits include reagents for use in the methods described herein, in one or more containers. Kits may include isotopically labeled biochemical precursors, as well as buffers, and/or excipients. Each reagent is supplied in a solid form or liquid buffer that is suitable for inventory storage, and later for exchange into a medium suitable for administration to a host organism in accordance with methods of the invention. Kits may also include means for administering the labeled biochemical precursors and/or means for obtaining one or more samples of a tissue or biological fluid from a living organism.

Kits are provided in suitable packaging. As used herein, “packaging” refers to a solid matrix or material customarily used in a system and capable of holding within fixed limits one or more of the reagent components for use in a method of the present invention. Such materials include glass and plastic (e.g., polyethylene, polypropylene, and polycarbonate) bottles, vials, paper, plastic, and plastic-foil laminated envelopes and the like.

Kits may optionally include a set of instructions in printed or electronic (e.g., magnetic or optical disk) form relating information regarding the components of the kits and their administration to a host organism and/or how to measure label incorporated into a biochemical component of an infectious agent. The kit may also be commercialized as part of a larger package that includes instrumentation for measuring isotopic content of a biochemical component, such as, for example, a mass spectrometer.

Information Storage Devices

The invention also provides for information storage devices such as paper reports or data storage devices comprising data collected from the methods of the present invention. An information storage device includes, but is not limited to, written reports on paper or similar tangible medium, written reports on plastic transparency sheets or microfiche, and data stored on optical or magnetic media (e.g., compact discs, digital video discs, optical discs, magnetic discs, and the like), or computers storing the information whether temporarily or permanently. The data may be at least partially contained within a computer and may be in the form of an electronic mail message or attached to an electronic mail message as a separate electronic file. The data within the information storage devices may be “raw” (i.e., collected but unanalyzed), partially analyzed, or completely analyzed. Data analysis may be by way of computer or some other automated device or may be done manually. The information storage device may be used to download the data onto a separate data storage system (e.g., computer, hand-held computer, and the like) for further analysis or for display or both. Alternatively, the data within the information storage device may be printed onto paper, plastic transparency sheets, or other similar tangible medium for further analysis or for display or both.

Isotopically-Perturbed Molecules

In another variation, the methods provide for the production of one or more isotopically-perturbed molecules (e.g., labeled fatty acids, lipids, carbohydrates, proteins, nucleic acids and the like) or one or more populations of isotopically-perturbed molecules. These isotopically-perturbed molecules comprise information useful in determining the flux of molecules within the metabolic pathways comprising the temporal and/or spatial gradients. Once isolated from a cell and/or a tissue of an organism, one or more isotopically-perturbed molecules are analyzed to extract information as described, supra.

### EXAMPLES

The following examples are intended to illustrate but not limit the invention.

### Example 1

### Livid Synthesis in Mouse Embryos

Female mice (Blk/6J) are administered 2% ²H₂O in drinking water starting one day prior to housing with male mice (one female and one male per cage). Female mice then become pregnant usually within 3 days. The drinking water content of ²H₂O is increased by 2% every 5 days (e.g., to 4% at day 5, 6% at day 10, and 8% at day 15). Urine is collected daily and ²H₂O content is measured by a gas chromatographic/mass spectrometric method.

On day 18-20, pregnant mice are sacrificed and the fetuses collected. Fat is extracted from visceral tissues and brain of fetuses, separated into triglycerides and phospholipids by thin layer chromatography, transesterified to fatty acid-methyl esters, and analyzed by GC/MS for isotope pattern. Predicted isotopic labeling patterns are calculated as described, supra, for example from tables prepared as described in the several MIDA references cited, supra, and previously incorporated by reference. The predicted isotopic labeling patterns are then compared with observed isotopic labeling patterns derived from actual measurements as described, supra, to temporally localize (i.e., establish the timing of) of lipid synthesis in the mouse embryos.

### Example 2

### Plasma Protein and Triglyceride Synthesis in Humans

Healthy human subjects are administered 70% ²H₂O orally for 4 weeks. The initial ²H₂O dosing regimen is 35 mL three times per day (morning, mid-day, and evening) for 4 days, then twice a day for 7 days, followed by once a day for 17 days. Urine is collected every 7 days for measurement of ²H₂O enrichment by GC/MS.

Blood samples are collected weekly. Plasma very-low-density lipoproteins (VLDL) are isolated by ultracentrifugation. Apolipoprotein B is precipitated from VLDL with heparin and hydrolyzed to free amino acids with 6N HCl in sealed tubes at 110° C. The amino acids are derivatized and analyzed by GC/MS. The ²H labeling pattern is measured for N-acetyl, N-butyl esters of glycine (m/z 174 and 175) and alanine (m/z 188 and 189). Lipids are extracted from VLDL and transesterified to fatty acid methyl esters. The free glycerol remaining after transesterification of acylglycerides is derivatized to glycerol triacetate. The ²H labeling pattern is measured by GC/MS for palmitate methyl ester (m/z 270-272) and glycerol triacetate (m/z 159-160) by GC/MS.

Predicted isotopic labeling patterns are calculated as described, supra, for the fragments of glycine, alanine, palmitate, and glycerol derivatives that were analyzed by GC/MS and compared with observed isotopic labeling patterns derived from actual measurements as described, supra, to temporally localize (i.e., establish the timing of) plasma protein and triglyceride synthesis. The measured isotopic fingerprints correlate with values of ²H₂O enrichment in the subject at the time the protein or triglyceride was synthesized. The predicted values are calculated for the entire range of ²H₂O enrichment in the temporal gradient of ²H₂O in the subject. The point on the gradient at which the isotopic fingerprint most strongly correlates with the predicted values represents the time that the synthesis occurred. Such data, in this example, can be used to determine when the synthesis of triglycerides or lipoproteins (critical components of the etiology of heart disease, a national epidemic) occur in a subject in response to a variety of inputs, including diet or therapy.

### Example 3

### DNA and Triglyceride Temporal Isotopic Gradient in Rat Tissues

Establishing a temporal gradient in vivo. A temporal gradient of a stable isotope labeled precursor (²H₂O) was established in rats as follows. Rats were given a bolus of 100% ²H₂O to give a body water value of 5% excess ²H₂O, then kept on 30% ²H₂O (via drinking water). Based on historical data, this regimen results in a steady increase of excess ²H₂O in body water from 5% on day 1 to a maximum of 15-18% at approximately day 4. Thus, a 4 day temporal gradient of 5 to 15% of ²H₂O was established in rats for this study.

Measuring the isotopic fingerprint. During the period of label administration (the 4 day temporal gradient) three animals were sacrificed on day 2 and three on day 4. From these animals, bone marrow and retroperitoneal fat pads were harvested. These samples were further processed: DNA was isolated from the bone marrow samples, and fat pads were separated into adipocyte (fat storing cells) and stromovascular (adipocyte supporting and precursor) cells. DNA was isolated from these two cell fractions. Additionally, total triglyceride was also isolated from the fat pads. These four isolated components (bone marrow DNA, retroperitoneal fat pad adipocyte DNA, retroperitoneal fat pad stromovascular cell DNA, and retroperitoneal fat pad tricglycerides) were processed and analyzed by GC/MS as described, supra (the isolation of these tissues, cells, their DNA, and triglycerides, and their analysis by GC/MS for de novo nucleotide synthesis or triglyceride synthesis are carried out using techniques well known in the art—see, e.g., U.S. Patent Application No. 60/581,028 herein incorporated by reference). For each component, the EM₁ and EM₂ values were determined from the GC/MS data. These values reflect the frequency of deuterium incorporation into either the ribose moiety of purine deoxynucleotides, or the glycerol moiety of triglycerides, and their ratio (EM₂/EM₁) reflects the concentration of the stable isotope precursor at the time that they were synthesized.

Calculating predicted isotopic fingerprints. Calculations were carried out to predict the EM₂/EM₁ ratio for ribose or glycerol for the range of body water enrichments in the gradient. These calculations were carried out as described, supra, and relied on the MIDA (combinatorial analysis) equations. A conceptual framework for these calculations is shown in FIG. 3. Calculations are made for every step of 0.5%, from 5 to 15%, and the output is expressed as a predicted ratio of EM₂/EM₁.

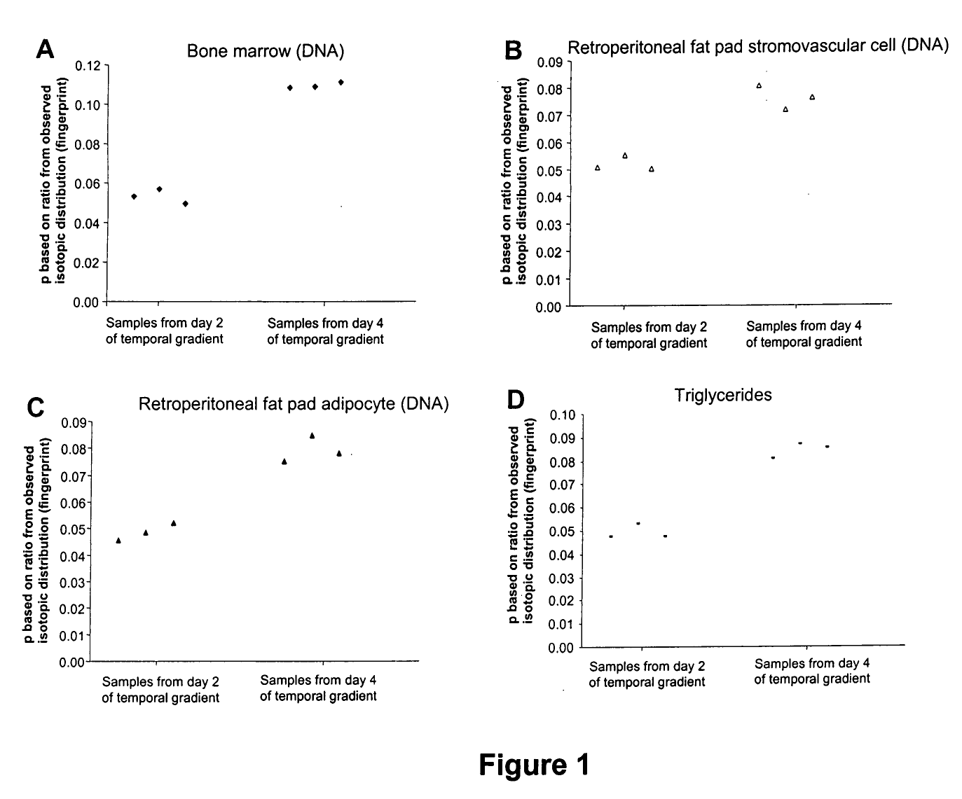

Comparison of actual and predicted values. Comparison of the measured values to the predicted values allows for the determination of when the analyzed sample was synthesized. The EM₂/EM₁ ratio in the measured sample is compared to the predicted values, and used to determine the concentration of ²H₂O at the time of synthesis (the value of excess ²H₂O used to calculate the most closely matching predicted ratio is taken as that from the time of synthesis). The actual values of excess ²H₂O resulting from such an analysis of the observed isotopic fingerprint are shown in FIG. 1. The values are 5.5% at day 2 and 11% for day 4 for the bone marrow DNA, 5% at day 2 and 8% for day 4 for both the stromovascular cell and adipocyte DNA, and 5% at day 2 and 8.5% for day 4 for the triglycerides.

Interpretation of the data. The observed isotopic fingerprints indicate that the synthesis begins immediately for all analytes (the excess ²H₂O values are 5% for day 2 samples, indicating that they were synthesized during the initial phase of the gradient, which began at 5%). The data further indicates that the fat pad synthesis occurred steadily over the gradient, as the day 4 samples reflect an excess ²H₂O of around 8%, which is less than the final value of the temporal gradient, which is closer to 15%. The bone marrow values at day 4, however, are higher, a result that reflects the more rapid replacement of bone marrow cells to adipose tissue. The value of 11% ²H₂O derived from the day 4 bone marrow sample indicates that while the components of the retroperitoneal fat pad were synthesized steadily over the course of the gradient, the bone marrow cells were synthesized more recently: analysis of their fingerprint places them further along the gradient (11% versus 8%).

Application of this example. This example was carried out in order to establish a model of fat pad (adipocyte/stromovascular cell/triglyceride) growth that can be used to rapidly evaluate the times of synthesis of these components in normal animals, and in response to a variety of stimuli, including drugs or dietary regimens. Shifts in the relative time of synthesis of triglycerides versus adipocyte DNA could, for instance, help distinguish between a drug that reduces triglyceride synthesis (a desired outcome—reducing fat accumulation) and a drug that simply suppresses adipocyte proliferation (not necessarily a desired outcome because each adipocyte can expand in size to accommodate more triglyceride). While other techniques can be used to determine these two parameters, this technique places the events in time, absolutely and with respect to each other, in the same animal, and it does so very rapidly—a significant improvement over stable isotope techniques that include no temporal gradient.

Effect of a therapeutic. Animals receiving thiozolidinedione treatment (at doses designed to prevent both fat tissue cell proliferation, i.e., no new DNA synthesized during drug treatment, and fat tissue triglyceride synthesis) did not show an isotopic temporal gradient as is depicted in FIG. 1.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention, which is delineated by the appended claims.

All publications, patents, and patent applications cited herein are hereby incorporated by reference in their entirety for all purposes to the same extent as if each individual publication, patent, or patent application were specifically and individually indicated to be so incorporated by reference.

1. A method of determining the timing of the synthesis of a biochemical component in a living organism, said method comprising:
(a) administering one or more stable isotopically-labeled biochemical precursors to an organism,
wherein the amount of one or more isotopically labeled biochemical precursors administered are varied over time to create a temporal gradient of isotopic enrichment in a biochemical precursor pool within the living organism, and
wherein the one or more isotopically labeled biochemical precursors are incorporated biosynthetically into one or more biochemical components of the living organism;
(b) obtaining one or more biological samples from the living organism, wherein said one or more biological samples comprise one or more biochemical components;
(c) measuring the isotopic labeling pattern in said one or more biochemical components; and
(d) comparing the isotopic labeling pattern measured in step (c) with a predicted isotopic labeling pattern across the temporal gradient or comparing isotopic labeling patterns in different biochemical components to determine the timing of biosynthesis of said biochemical component.

2. The method of claim 1, wherein the administering step (a) comprises increasing the amount of said one or more isotopically labeled biochemical precursors over time.

3. The method of claim 1, wherein said administering step (a) comprises decreasing the amount of said isotopically labeled biochemical precursors administered over time.

4. The method of claim 1, wherein said administering step (a) comprises administering a plurality of isotopically labeled biochemical precursors, wherein the amount of at least one of said isotopically labeled biochemical precursors is increased over time and the amount of at least one of said isotopically labeled biochemical precursors is decreased over time.

5. The method of claim 1, wherein said isotopic label is chosen from ²H, ¹³C, ¹⁵N, and ¹⁸O.

6. The method of claim 5, wherein said isotopic label is ²H.

7. The method of claim 1, wherein said biochemical precursor is chosen from amino acids, monosaccharides, lipids, CO₂, NH₃, H₂O, nucleosides, and nucleotides.

8. The method of claim 1, wherein said biochemical component is chosen from polypeptides, polynucleotides, purines, pyrimidines, amino acids, carbohydrates, lipids, and porphyrins.

9. The method of claim 1, wherein the living organism is a prokaryotic cell.

10. The method of claim 1, wherein the living organism is a eukaryotic cell.

11. The method of claim 1, wherein the living organism is a mammal.

12. The method of claim 11, wherein the mammal is a human.

13. The method of claim 1, wherein the biological sample is collected at the termination of a biological process of interest.

14. The method of claim 1, wherein a plurality of biochemical components is isolated and the isotopic labeling patterns of said biochemical components are compared to one another to establish their relative timing of biosynthesis.

15. The method of claim 1, wherein the isotopic labeling pattern is determined by mass spectrometry or NMR spectroscopy.

16. A method for determining the spatial localization of a biosynthetic event in a living organism, said method comprising:
(a) administering at least one biochemical precursor comprising a detectable amount of an isotopic label,
wherein the amount of isotopic label administered varies spatially within the living organism to create a spatial gradient of isotopic enrichment in a biochemical precursor pool within the living organism, and
wherein the at least one biochemical precursor is incorporated biosynthetically into one or more biochemical components of the living organism;
(b) isolating the one or more biochemical components from a biological sample of the living organism;
(c) determining the isotopic labeling pattern in the one or more biochemical components; and
(d) establishing the spatial location of biosynthesis of the one or more biochemical components by comparing the isotopic labeling pattern determined in step (c) with predicted isotopic labeling patterns across the spatial gradient or by comparing isotopic labeling patterns in different biochemical components.

17. The method of claim 16, wherein said isotopic label is chosen from ²H, ¹³C, ¹⁵N, and ¹⁸O.

18. The method of claim 17, wherein said isotopic label is ²H.

19. The method of claim 16, wherein said at least one biochemical precursor is chosen from amino acids, monosaccharides, lipids, CO₂, NH₃, H₂O, nucleosides, and nucleotides.

20. The method of claim 16, wherein said one or more biochemical components is chosen from polypeptides, polynucleotides, purines, pyrimidines, amino acids, carbohydrates, lipids, and porphyrins.

21. The method of claim 16, wherein the living organism is a mammal.

22. The method of claim 21, wherein the mammal is a human.

23. The method of claim 16, wherein the biological sample is collected at the termination of a biological process of interest.

24. The method of claim 16, wherein a plurality of biochemical components are isolated and the isotopic labeling patterns of said plurality of biochemical components are compared to one another to establish their relative spatial location of biosynthesis.

25. An information storage device comprising data obtained from the method according to claim 1.

26. An information storage device comprising data obtained from the method according to claim 16.

27. The device of claim 25, wherein said device is a printed report.

28. The printed report of claim 27, wherein the medium in which said report is printed on is chosen from paper, plastic, and microfiche.

29. The device of claim 25, wherein said device is a computer disc.

30. The disc of claim 29, wherein said disc is chosen from a compact disc, a digital video disc, an optical disc, and a magnetic disc.

31. An isotopically-perturbed molecule generated by the method according to claim 1.

32. The isotopically-perturbed molecule of claim 31, wherein said molecule is chosen from protein, lipid, nucleic acid, glycosaminoglycan, proteoglycan, porphyrin, and carbohydrate molecules.

33. An isotopically-perturbed molecule generated by the method according to claim 16.

34. The isotopically-perturbed molecule of claim 33, wherein said molecule is chosen from protein, lipid, nucleic acid, glycosaminoglycan, proteoglycan, porphyrin, and carbohydrate molecules.
